(19) United States
(12) Patent Application Publication
(10) Pub. No.: US 2004/0141943 A1
(43) Pub. Date: Jul. 22, 2004

(54) Novel cationic associative polyurethanes and their use

(75) Inventors: Nathalie Mougin, Paris (FR); Francois Cottard, Levallois-Perret (FR); Roland De La Mettrie, Le Vesinet (FR); Bertrand Lion, Luzarches (FR); Elise Maury, Paris (FR)

Correspondence Address: PILLSBURY WINTHROP, LLP, P.O. BOX 10500, MCLEAN, VA, 22102, US

(73) Assignee: L'Oreal, Paris (FR)

(21) Appl. No.: 10/751,514

(22) Filed: Jan. 6, 2004

Related U.S. Application Data
(62) Division of application No. 09/904,516, filed on Jul. 16, 2001.

(30) Foreign Application Priority Data
Jul. 21, 2000 (FR) 0009609

(51) Int. Cl. A61K 7/06; A61K 7/11; C08G 18/28; C08G 18/70

(52) U.S. Cl. 424/70.17; 528/71

(57) ABSTRACT

The invention relates to water-soluble amphiphilic cationic associative polyurethanes of formula R—X—(P)ₙ[L-(Y)ₘ]ᵣ-L′-(P′)ₚ—X′—R′, the molecule comprising at least one protonated or quaternized amine functional group and at least one hydrophobic group. The invention also relates to the use of these polyurethanes as thickeners or gelling agents in cosmetic compositions for topical application.

### CROSS-REFERENCE TO RELATED APPLICATIONS

This is a divisional of U.S. patent application Ser. No. 09/904,516, filed Jul. 16, 2001, which claims priority to French Patent Appl. No. 0009609, filed Jul. 21, 2000, the contents of both applications are herein incorporated in their entirety by reference.

### DETAILED DESCRIPTION

The present invention relates to novel water-soluble or water-dispersible amphiphilic cationic associative polyurethanes and to their use in compositions for topical application, in particular for cosmetic or therapeutic use.

The term “water-soluble” relating to the associative polyurethanes of the present invention means that these polymers have a solubility in water at ambient temperature at least equal to 1% by weight, that is to say that, up to this concentration, no precipitate can be detected with the naked eye and the solution is perfectly clear and homogeneous.

The term “water-dispersible” polyurethanes is understood to mean polymers which, when they are suspended in water, spontaneously form globules having a mean size, measured by light scattering on a device of Coulter type, of between 5 nm and 600 nm and in particular between 5 nm and 500 nm.

The thickening and/or the gelling of aqueous media by polymers has been an important subject of research for a long time, in particular in the field of cosmetics and pharmaceuticals. The production of an advantageous thickening effect by water-soluble polymer generally supposes a high molar mass and a high hydrodynamic volume. The gelling of an aqueous medium is then regarded as the result of a three-dimensional polymer network obtained by a crosslinking of linear polymers or by copolymerization of bifunctional and polyfunctional monomers. However, the use of such polymers of very high molar mass presents a number of problems, such as the not very pleasant texture of and the difficulty spreading the gels obtained.

One advantageous approach has consisted in using, as thickener, polymers capable of reversibly associating with one another or with other molecules or particles. This physical association gives rise to thixotropic or shear-thinning macromolecular systems, that is to say systems for which the viscosity depends on the shear forces to which they are subject.

Such polymers, capable of reversibly associating with one another or with other molecules, are known as “associative polymers”. The interaction forces involved can be very different in nature, for example of electrostatic nature, of hydrogen bond type or hydrophobic interactions.

A specific case of associative polymers is amphiphilic polymers, that is to say polymers comprising one or more hydrophilic parts, which render them soluble in water, and one or more hydrophobic regions, by which the polymers interact and combine with one another or with other molecules.

The use of associative polymers and in particular of associative polyurethanes has already been recommended in cosmetics. However, the Theological and cosmetic properties of these polymers are not optimum.

A novel family of water-soluble or water-dispersible amphiphilic cationic associative polyurethanes has been discovered, which polyurethanes have excellent thickening qualities and possess good cosmetic properties.

Its excellent thickening properties allow the polymer to be used in a smaller amount. This advantage makes it possible to improve the texture of the composition comprising it.

The gel obtained by using the associative polyurethanes of the invention is pleasant to the touch and spreads easily.

A subject-matter of the present invention is thus a novel family of water-soluble or water-dispersible amphiphilic cationic associative polyurethanes.

Another subject-matter of the present invention is a cosmetic composition comprising at least one water-soluble or water-dispersible amphiphilic cationic associative polyurethane.

A third subject-matter of the invention is the use of these polyurethanes as thickeners or gelling agents in compositions for topical application with a cosmetic or therapeutic use.

Other subject matters will become apparent on reading the description and examples which follow.

The family of water-soluble or water-dispersible amphiphilic cationic associative polyurethanes in accordance with the invention can be represented by the following general formula (I):

R—X—(P)ₙ-L-(Y)ₘ]ᵣ-L′-(P′)ₚ—X′—R′ (I)

in which:

R and R′, which are identical or different, represent a hydrophobic group or a hydrogen atom;

X and X′, which are identical or different, represent a group comprising an amine functional group which may or may not carry a hydrophobic group or the L″ group;

L, L′ and L″, which are identical or different, represent a group derived from a diisocyanate;

P and P′, which are identical or different, represent a group comprising an amine functional group which may or may not carry a hydrophobic group;

Y represents a hydrophilic group;

r is an integer between 1 and 100, preferably between 1 and 50 and in particular between 1 and 25,

n, m, and p have values, each independently of the others, between 0 and 1000;

the molecule comprising at least one protonated or quaternized amine functional group and at least one hydrophobic group.

In a preferred embodiment of the polyurethanes of the present invention, the only hydrophobic groups are the R and R′ groups at the chain ends.

A preferred family of cationic associative polyurethanes according to the present invention is that corresponding to the formula I above in which:

R and R′ both independently represent a hydrophobic group,

X and X′ each represent an L″ group,

n and p have values between 1 and 1000 and

L, L′, L″, P, P′, Y and m have the meaning indicated above.

Another family of cationic associative polyurethanes according to the present invention is that corresponding to the formula I above in which R and R′ both independently represent a hydrophobic group, X and X′ each represent an L″ group, n and p have the value 0 and L, L′, L″, Y and m have the meaning indicated above.

The fact that n and p have the value 0 means that these polymers do not comprise units derived from a monomer with an amine functional group incorporated in the polymer during the polycondensation. The protonated amine functional groups of these polyurethanes result from the hydrolysis of excess isocyanate functional groups at the chain end, followed by the alkylation of the primary amine functional groups formed by alkylating agents comprising a hydrophobic group, that is to say compounds of RQ or R′Q type in which R and R′ are as defined above and Q denotes a leaving group, such as a halide, a sulphate, and the like.

Yet another preferred family of cationic associative polyurethanes according to the present invention is that corresponding to the formula I above in which:

R and R′ both independently represent a hydrophobic group,

X and X′ both independently represent a group comprising a quaternary amine,

n and p have the value zero, and

L, L′ and Y and m have the meaning indicated above.

The number-average molecular mass of the amphiphilic cationic associative polyurethanes of the invention is preferably between 400 and 500,000, in particular between 1,000 and 400,000 and ideally between 1,000 and 300,000.

The term “hydrophobic group” is understood to mean a radical or polymer with a saturated or unsaturated and linear or branched hydrocarbonaceous chain which can comprise one or more heteroatoms, such as P, O, N or S, or a radical with a perfluorinated or silicone chain. When it denotes a hydrocarboneous radical, the hydrophobic group comprises at least 10 carbon atoms, preferably from 10 to 30 carbon atoms, in particular from 12 to 30 carbon atoms and more preferably from 18 to 30 carbon atoms.

The hydrocarbonaceous hydrophobic group preferably originates from a monofunctional compound.

By way of examples, the hydrophobic group can result from a fatty alcohol, such as stearyl alcohol, dodecyl alcohol or decyl alcohol. The hydrophobic group can also be a hydrocarbonaceous polymer, such as, for example, polybutadiene:

When X and/or X′ denote a group comprising a tertiary or quaternary amine, X and/or X′ can represent one of the following formulae:

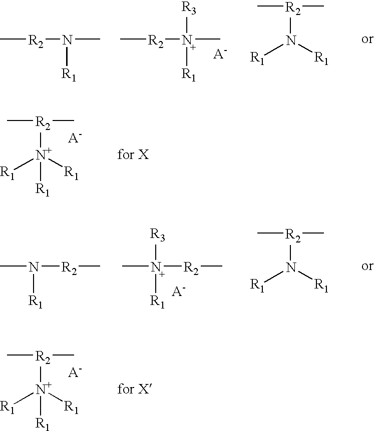

in which:

R₂ represents a linear or branched alkylene radical having from 1 to 20 carbon atoms, which may or may not comprise a saturated or unsaturated ring, or an arylene radical, it being possible for one or more carbon atoms to be replaced by a heteroatom chosen from N, S, O, or P;

R₁ and R₃, which are identical or different, denote a linear or branched C₁-C₃₀ alkyl or alkenyl radical or an aryl radical, it being possible for at least one of the carbon atoms to be replaced by a heteroatom chosen from N, S, O or P;

A⁻ is a physiologically acceptable counter ion.

The L, L′ and L″ groups represent a group of formula:

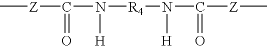

in which:

Z represents —O—, —S— or —NH—; and

R₄ represents a linear or branched alkylene radical having from 1 to 20 carbon atoms, which may or may not comprise a saturated or unsaturated ring, or an arylene radical, it being possible for one or more of the carbon atoms to be replaced by a heteroatom chosen from N, S, O, and P.

The P and P′ groups, comprising an amine functional group, can represent at least one of the following formulae:

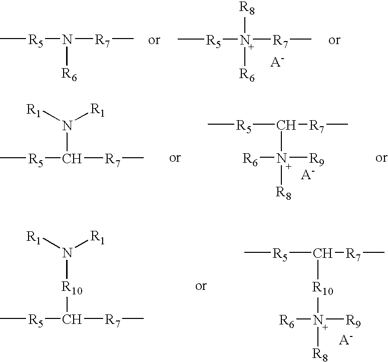

in which:

R₅ and R₇ have the same meanings as R₂ defined above;

R₆, R₈, and R₉ have the same meanings as R₁ and R₃ defined above;

R₁₀ represents a linear or branched alkylene group which is optionally unsaturated and which can comprise one or more heteroatoms chosen from N, O, S, and P;

and A⁻ is a physiologically acceptable counter ion.

As regards the meaning of Y, the term “hydrophilic group” is understood to mean a water-soluble polymeric or non-polymeric group.

Mention may be made, by way of examples, when polymers are not concerned, of ethylene glycol, diethylene glycol and propylene glycol.

When, in accordance with a preferred embodiment of the invention, a hydrophilic polymer is concerned, mention may be made, by way of examples, of polyethers, sulphonated polyesters, sulphonated polyamides or a mixture of these polymers. The hydrophilic compound is preferably a polyether and in particular a poly(ethylene oxide) or poly(propylene oxide).

The amphiphilic cationic associative polyurethanes of formula I according to the invention are formed from diisocyanates and various compounds possessing functional groups with a labile hydrogen. The functional groups with a labile hydrogen can be alcohol, primary or secondary amine, or thiol functional groups, giving, after reaction with the diisocyanate functional groups, respectively polyurethanes, polyureas and polythioureas. The term <<polyurethanes>> chosen to denote the novel associative polymers of the present invention encompasses these three types of polymers, namely polyurethanes proper, polyureas and polythioureas, and copolymers of these.

A first type of compound participating in the preparation of the polymer of formula I of the invention is a compound comprising at least one unit with an amine functional group. This compound can be multifunctional but the compound is preferably difunctional, that is to say that, according to a preferred embodiment of the invention, this compound comprises two labile hydrogen atoms carried, for example, by a hydroxyl, primary amine, secondary amine or thiol functional group. Use may also be made of a mixture of multifunctional and difunctional compounds in which the percentage of multifunctional compounds is low.

As indicated above, this compound can comprise more than one unit with an amine functional group. It is then a polymer carrying a repetition of the unit with an amine functional group.

This type of compound can be represented by one of the following formulae:

HZ-(P)ₙ-ZH,

or

HZ-(P′)ₚ-ZH

in which Z, P, P′, n and p are as defined above.

Mention may be made, as examples of a compound with an amine functional group, of N-methyldiethanolamine, N-(tert-butyl)diethanolamine or N-sulpho-ethyldiethanolamine.

The second compound participating in the preparation of the polymer of formula I according to the invention is a diisocyanate corresponding to the formula:

O═C═N—R₄—N═C═O

in which R₄ is defined above.

Mention may be made, by way of examples, of methylenediphenyl diisocyanate, methylenecyclohexane diisocyanate, isophorone diisocyanate, toluene diisocyanate, naphthalene diisocyanate, butane diisocyanate or hexane diisocyanate.

A third compound participating in the preparation of the polymer of formula I according to the invention is a hydrophobic compound intended to form the end hydrophobic groups of the polymer of formula I.

This compound is composed of a hydrophobic group and of a functional group with a labile hydrogen, for example a hydroxyl, primary or secondary amine, or thiol functional group.

By way of examples, this compound can be a fatty alcohol, such as, in particular, stearyl alcohol, dodecyl alcohol or decyl alcohol. When this compound comprises a polymer chain, it can be, for example, a-hydroxyl hydrogenated polybutadiene.

The hydrophobic group of the compound of formula I according to the invention can also result from the quaternization reaction of the tertiary amine of the compound comprising at least one tertiary amine unit. Thus, the hydrophobic group is introduced by the quaternizing agent. This quaternizing agent is a compound of RQ or R′Q type in which R and R′ are as defined above and Q denotes a leaving group, such as a halide, a sulphate, and the like.

The amphiphilic cationic associative polymer of the invention can additionally comprise a hydrophilic sequence. This sequence is contributed by a fourth type of compound participating in the preparation of the polymer. This compound can be multifunctional. It is preferably difunctional. It is also possible to have a mixture where the percentage of multifunctional compound is low.

The functional groups with the labile hydrogen are alcohol, primary or secondary amine, or thiol functional groups. This compound can be a polymer terminated at the ends of the chains by one of these functional groups with a labile hydrogen.

Mention may be made, by way of examples, when polymers are not concerned, of ethylene glycol, diethylene glycol and propylene glycol.

When a hydrophilic polymer is concerned, mention may be made, by way of examples, of polyethers, sulphonated polyesters, sulphonated polyamides or a mixture of these polymers. The hydrophilic compound is preferably a polyether and in particular a poly(ethylene oxide) or poly (propylene oxide).

The polymer prepared from the compounds defined above is an amphiphilic cationic associative polymer of formula I according to the present invention. This polymer is soluble or dispersible in water and spectacularly increases the viscosity of the aqueous solution in which it is dissolved or dispersed.

The hydrophilic group, recorded as Y in the formula I, is optional. This is because the units with a quaternary or protonated amine functional group may suffice to contribute the dispersibility in water or the solubility necessary for this type of polymer in an aqueous solution.

Although the presence of a hydrophilic Y group is optional, amphiphilic cationic associative polyurethanes comprising such a group are preferred, however.

Given its good thickening properties and its excellent affinity for keratinous substances, this type of amphiphilic cationic associative polymer according to the invention is particularly suitable for the preparation of compositions intended for topical application with a cosmetic use.

In particular, the polymers according to the invention can be used in hair compositions, in compositions for caring for the skin, in compositions for caring for the nails, in scenting compositions and in compositions for making up the skin, lips, eyelashes and nails.

Amphiphilic cationic associative polymers of formula I, illustrating the invention, follow below by way of examples:

### EXAMPLE 1

The following polymer:

C₁₈H₃₇—O—CONHR₄NHCO—O—(CH₂)₂—N⁺(CH₃)(CH₃)—(CH₂)₂—O—CONHR₂NHCO—O (PEO) O—CONHR₂NHCO—O—(CH₂)₂—N⁺(CH₃)(CH₃)—(CH₂)₂—O—CONHR₄NHCO—OC₁₈H₃₇

with:

counter ion: CH₃SO₄⁻

R₄=methylenedicyclohexyl

is synthesized from the following reactants:

| | |
|---|---|
| C₁₈H₃₇OH | 2 mol |
| Methylenedicyclohexyl diisocyanate | 4 mol |
| Polyethylene glycol | 1 mol |
| N-methylethanolamine | 2 mol |
| Quaternizing agent (CH₃)₂SO₄ | 2 mol |

### EXAMPLE 2

The following polymer:

C₁₈H₃₇N⁺(CH₃)(CH₃)—(CH₂)₂—O—CONHR₄NHCO—O (PEO) O—CONHR₄NHCO—O—(CH₂)₂—N⁺(CH₃)(CH₃)C₁₈H₃₇

with:

R₄=methylenedicyclohexyl

Counter ion: Cl

is synthesized from the following reactants:

| | |
|---|---|
| Methylenedicyclohexyl diisocyanate | 2 mol |
| Polyethylene glycol | 1 mol |
| N,N-Dimethylethanolamine | 2 mol |
| Quaternizing agent C₁₈H₃₇OH | 2 mol |

### EXAMPLE 3

Reactants:

| Poly(ethylene oxide) (PEG) (Mₙ 10,000): | 0.010 mol |
|---|---|
| Methylenedicyclohexyl diisocyanate: | 0.018 mol |
| N,N-Dimethylethanolamine: | 0.020 mol |
| Stearyl bromide: | 0.024 mol |
| Tin octanoate (catalyst): | 0.2% |

0.010 mol (100 g) of poly(ethylene oxide) (PEG), having a number-average mass of 10,000, is dissolved in 105 g of THF comprising 0.2% of tin octanoate (catalyst) and then 0.018 mol (4.71 g) of methylenedicyclohexyl diisocyanate is added drop wise. The reaction medium is heated for 15 hours at reflux of the THF, 100 ml of THF being added after 6 hours. During the reaction, partial disappearance of the NCO band of the isocyanate and the appearance of the CO and NH bands of the amide bonds formed are observed by FTIR. The medium is very viscous and transparent.

0.020 mol (1.78 g) of N,N-dimethylethanolamine is subsequently added and the reaction is allowed to continue for 4 hours at reflux of the THF until the NCO band and the OH band of the alcohol have completely disappeared.

For the quaternization, 0.024 mol (8 g) of stearyl bromide, that is to say an excess of 20% in moles with respect to the N,N-dimethylethanolamine, is added to the reaction mixture, followed by 100 g of THF, to thin the very viscous reaction medium. Heating is continued at reflux of the THF for an additional 36 hours.

The polymer obtained is precipitated from petroleum ether, is filtered off and is dried under vacuum at 40° C. for 24 hours. A friable white powder is thus obtained.

A number-average mass of 70,000 and a weight-average mass of 115,000, which corresponds to a polydispersity index of 1.65, are measured by gel permeation chromatography in an aqueous medium (calibration with polystyrene).

### EXAMPLE 4

Reactants:

| Poly(ethylene oxide) (PEG) (Mₙ 10,000): | 0.010 mol |
|---|---|
| Methylenedicyclohexyl diisocyanate: | 0.020 mol |
| Water: | 0.0334 mol |
| Stearyl bromide: | 0.022 mol |
| Dibutyltin dilaurate (catalyst): | 0.5% |

100 g of polyethylene glycol are dried overnight in an oven at a temperature of 100° C. in the presence of P₂O₅. The next day, PEG is heated with mechanical stirring at a temperature of 100° C. while flushing with argon in order to remove any residual trace of water. This operation lasts 1 hour and 30 minutes. 0.53 g of dibutyltin dilaurate (catalyst) is subsequently added drop wise to the PEG. After 10 minutes, 0.020 mol (5.24 g) of methylenedicyclohexyl diisocyanate is added and reaction takes place for 1 hour and 30 minutes. 105 g of toluene are added to reduce the high viscosity of the medium. After reacting for 22 hours, 0.15 g of methylenedicyclohexyl diisocyanate is added to the reaction mixture and the reaction is continued for a further 2 hours.

0.0334 mol (6 g) of water is subsequently added and the reaction is continued for about 8 hours, 105 g of toluene being added 3 times after reacting for 2 hours, 4 hours, and 6 hours respectively. At the end of the reaction, the toluene is evaporated.

For the alkylation of the primary amine functional groups resulting from the hydrolysis of the isocyanate groups at the ends of the polymer, the polymer prepared above is dissolved in 105 g of THF, so as to obtain a 50% by weight solution, and 0.022 mol (7.33 g) of. stearyl bromide is added. The mixture is heated at reflux of the THF for 20 hours. The polymer is isolated by precipitation from petroleum ether, filtration and drying under vacuum at 40° C. for 24 hours. A friable white powder is thus obtained.

A number-average mass of 52,000 and a weight-average mass of 108,000, which corresponds to a polydispersity index of 2.07, are measured by gel permeation chromatography in an aqueous medium (calibration with polystyrene).

What is claimed is:

1. Water-soluble amphiphilic cationic associative polyurethanes of formula (I):
R—X—(P)ₙ-[L-(Y)ₘ]ᵣ-L′-(P′)ₚ—X′—R′ (I)
in which:
R and R′, are identical or different, and represent a hydrophobic group or a hydrogen atom;
X and X′, are identical or different, and represent a group comprising an amine functional group which may or may not carry a hydrophobic group or an L″ group;
L, L′ and L″, are identical or different, and represent a group derived from diisocyanate;
P and P′, are identical or different, and represent a group comprising an amine functional group which may or may not carry a hydrophobic group;
Y represents a hydrophilic group;
r is an integer between 1 and 100,
n, m and p have values, each independently of the others, between 0 and 1000;
the molecule comprising at least one protonated or quaternized amine functional group and at least one hydrophobic group.

2. The polyurethane according to claim 1, wherein the only hydrophobic groups are the R and R′ groups.

3. The polyurethane according to claim 1, wherein R and R′ independently represent a hydrophobic group; X and X′ are L″; n and p have values between 1 and 1000; and L, L′, L″, P, P′, Y and m are the same as claim 1.

4. The polyurethane according to claim 1, wherein R and R′ independently represent a hydrophobic group; X and X′ are L″; n and p have the value 0; and L, L′, L″, Y and m are the same as claim 1.

5. The polyurethane according to claim 1, wherein R and R′ independently represent a hydrophobic group; X and X′ comprise a quartenary amine; n and p have the value 0; and L, L′, Y and m are the same as claim 1.

6. The polyurethane according to claim 1, which exhibits a number-average molecular mass between 400 and 500,000.

7. The polyurethane according to claim 6, which has a number-average molecular mass ranging from 1,000 to 400,000.

8. The polyurethane according to claim 7, which has a number-average molecular mass ranging from 1,000 to 300,000.

9. The polyurethane according to claim 1, wherein R and R′ represent a radical or a polymer with a saturated or unsaturated and linear or branched hydrocarbonaceous chain, in which chain one or more of the carbon atoms is optionally replaced by a heteroatom selected from the group consisting of S, N, O and P, or a radical comprising a silicone or perfluorinated chain.

10. The polyurethane according to claim 1, wherein X and X′ represent one of the formulae: 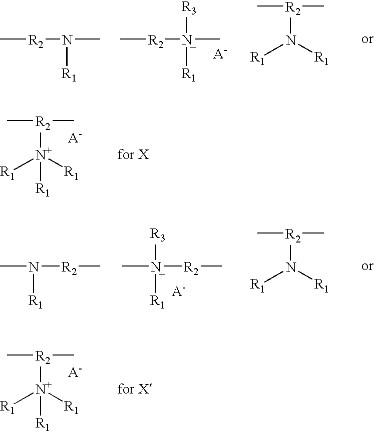
in which:
R₂ represents a linear or branched alkylene radical having from 1 to 20 carbon atoms, which optionally may comprise a saturated or unsaturated ring, or an arylene radical, wherein one or more carbon atoms optionally is replaced by a heteroatom selected from the group consisting of N, S, O, or P;
R₁ and R₃, are identical or different, are a linear or branched C₁-C₃₀ alkyl or alkenyl radical or an aryl radical, wherein at least one of the carbon atoms optionally can be replaced by a heteroatom selected from the group consisting of N, S, O, and P;
A⁻ is a physiologically acceptable counter ion.

11. The polyurethane according to claim 1, wherein L, L′, and L″ are identical or different, represent the formula: 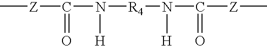
in which:
Z represents —O—, —S—, or —NH—; and
R₄ represents a linear or branched alkylene radical having from 1 to 20 carbon atoms, which optionally may comprise a saturated or unsaturated ring, or an arylene radical, wherein one or more of the carbon atoms optionally is replaced by a heteroatom chosen from N, S, O, and P.

12. The polyurethane according to claim 1, wherein P and P′ are identical or different, and are selected from the following formulae: 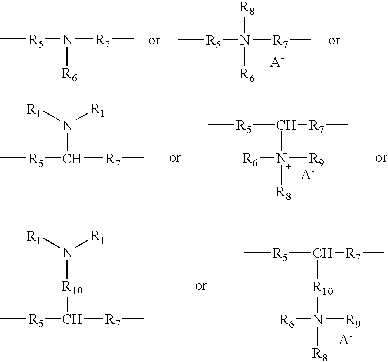
R₅ and R₇ are identical or different and represents a linear or branched alkylene radical having from 1 to 20 carbon atoms, which optionally may comprise a saturated or unsaturated ring, or an arylene radical, wherein one or more carbon atoms optionally is replaced by a heteroatom selected from the group consisting of N, S, O, or P;
R₆, R₈ and R₉ are identical or different, are a linear or branched C₁-C₃₀ alkyl or alkenyl radical or an aryl radical, wherein at least one of the carbon atoms optionally can be replaced by a heteroatom selected from the group consisting of N, S, Om and P;
R₁₀ represents a linear or branched alkylene group which is optionally unsaturated and which optionally comprises one or more heteroatoms selected from the group consisting of N, O, S and P, and
A⁻ is a physiologically acceptable counter ion.

13. The polyurethane according to claim 1, wherein Y represents a glycol selected from the group consisting of ethylene glycol, diethylene glycol and propylene glycol or a polymer selected from the group consisting of polyethers, sulphonated polyesters and sulphonated polyamides.

14. A method for using a polyurethane as defined in claim 1 as a thickener or gelling agent comprising adding said polyurethane to a composition which is to be used for topical application as a cosmetic.

15. A cosmetic composition thickened or gellified with at least one water-soluble polyurethane according to claim 1.

16. The polyurethane according to claim 1, wherein r is an integer between 1 and 50.

17. The polyurethane according to claim 16, wherein r is an integer between 1 and 25.
